(19) United States
(12) Patent Application Publication
(10) Pub. No.: US 2010/0221330 A1
(43) Pub. Date: Sep. 2, 2010

(54) BUOYANT FORMULATIONS OF BETAINE

(76) Inventor: Jallal Messadek, Liege (BE)

Correspondence Address: HOVEY WILLIAMS LLP, 10801 Mastin Blvd., Suite 1000, Overland Park, KS, 66210, US

(21) Appl. No.: 12/704,294

(22) Filed: Feb. 11, 2010

Related U.S. Application Data
(63) Continuation of application No. 11/251,737, filed on Oct. 17, 2005, now abandoned.
Application No. 11/251,737 is a continuation-in-part of application No. PCT/BE2004/000053, filed on Apr. 18, 2004.

(30) Foreign Application Priority Data
Apr. 17, 2003 (BE) BE2003/0248

(51) Int. Cl. A61K 31/205; A61K 9/50; A61K 9/48; A61K 9/00; A61K 31/616; A61K 31/4365; A61K 9/14; A61P 9/00; A61P 9/10; A61P 9/12; A61P 7/02; A61P 25/28; A61P 25/16; A61P 3/04; A61P 35/00; A61P 17/06; A61P 29/00; A61P 15/00; A61P 19/00; A61P 25/00; A61P 1/00

(52) U.S. Cl. 424/463; 514/556; 424/490; 424/492; 424/400; 514/162; 514/301; 424/484; 977/773; 977/906

(57) ABSTRACT

An oral control release formulation for releasing at least one betaine after oral administration to a human, said formulation comprising at least one pharmaceutically acceptable means ensuring an at least partial floating of the formulation releasing at least one betaine in the gastro-intestinal tractus.

This application is a continuation of pending U.S. patent application Ser. No. 11/251,737 entitled BUOYANT FORMULATIONS OF BETAINE, and filed Oct. 17, 2005, which is a continuation-in-part application under 35 U.S.C. 111 of PCT/BE2004/000053 filed on Apr. 18, 2004, published on Oct. 28, 2004, under number WO2004/091601, and claiming the priority of Belgian Patent Application BE2003/0248 filed on Apr. 17, 2003. Each of the above applications is incorporated by reference herein.

### ABSTRACT OF THE DISCLOSURE

The present invention relates to a floating controlled release formulation or dosage form intended to release at least one betaine after oral administration. The oral administration of a betaine contained in a solid, semi-solid or liquid pharmaceutical form for controlled release enables to obtain the intended blood levels and to maintain them stable for a long period. The aim of the present invention is a floating matrix form destined for increasing gastric residence average time for at least one betaine in order to increase its bioavailability, to increase the passage of said betaine in the blood and to allow an extended control of plasmatic profiles. The aim of the present invention is also a process and/or a system enabling to realize at least one or more of the following points:
a) to increase the residence time in the gastro-intestinal tractus of at least one betaine
b) to control and/or to increase in function of time the release of at least one betaine in the blood circulation
c) to control and/or to increase the release amount of at least one betaine in the blood circulation
d) to control and/or to increase the bioavailability of at least one betaine

The invention further relates to a method of treatment of a patient suffering from a trouble or a method for preventing a patient from suffering a trouble, in which an efficient amount of at least one betaine is administered orally by means of a floating controlled release formulation.

### STATE OF THE ART

Betaines (CH₃)₃N⁺—(CH₂)ₙ—COO⁻, n being an integer of 1 to 5, are molecules known for their osmoprotectant properties, as well as their cosmetic and pharmaceutical uses. Various pharmaceutical uses are known, and in particular the use of betaine for the treatment of homocystinuria. Homocystinuria is due to high homocysteine levels in the plasma of the affected patients. The administration of betaine enables to reduce the homocysteine concentration in the blood. About the half of homocystinuric patients are treated with high oral doses of betaine of 6 g to 20 g per day. Such high doses of betaine are necessary for achieving patient plasmatic concentrations of about 200 to 400 μMol/1 (An indirect of homocysteine suppression by betaine: optimizing the dosage regimen of betaine in homocystinuria, Angela Matthews et al, Br J Clin Pharmacol, 54, 140-146).

After oral administration, the total bioavailability of betaine appears to be very low, of about 10 to 15% (Schwahn B C et al: Pharmacokinetic of oral betaine in healthy subjects and patients with homocystinuria. Br J Clin Pharmacol 2003 January; 55(,1): 6-13).

Betaine for treating homocystinuria is available in the form of a drug CYSTADANE®. The usual dosage for adult patients and young patients is 6 grams per day, orally administered, in fractionated doses of 3 grams, twice a day. The prescribed amount of CYSTADANE® (Betaine anhydrous powder for oral solution) has to be calculated by means of a furnished measuring spoon (The spoon with single volume of 1.7 cc is equal to 1 gram of anhydrous betaine powder) and then dissolved in 4 to 6 ounces of water, juice, milk or maternal milk or mixed with food for immediate intake (CYSTADANE® monograph). No other oral administration methods are known for the betaine and it appears that the prescribed high amounts (up to 20 grams per day) show the low bioavailability of betaine.

When examining the plasmatic profiles of betaine after oral administration to humans, it appears that some betaine (i.e. a small amount) passes quickly in the blood so as to be quickly distributed in various organs. This quick drop of the betaine level on the blood is not desired as its major action sites are located in the circulation. On the other hand, betaine is naturally present in physiological liquids and is not xenobiotic. Having a low molecular weight and being naturally present in the body, a better bioavailability could be expected. Furthermore, studies relating to the absorption site of betaine in the gastro-intestinal tractus show that betaine is absorbed by the duodenum and the jejunum (H. Kettunen and al., Intestinal uptake of betaine in vitro and the distribution of methyl groups from betaine, choline, and methionine in the body of broiler chicks. Comparative Biochemistry and Physiology Part A 128 2001 269-278).

It seems thus that the cause of betaine low oral bioavailability at intestinal level is well due to a passage essentially limited at the level of the parts the most adjacent to the small intestine. Another limiting cause could be the intestinal saturation, causing the fact that betaine above a determined level can no more be absorbed. Then, the use of floating forms seems well the best possible path for optimizing the intestinal intake of this substance. The other path would consist to increase betaine adhesion on the gastro intestinal tractus so as to extend its residence inside the tractus.

### AIM OF THE INVENTION

In view of the remarkable pharmacological properties of betaines, especially discovered by the inventor and disclosed in U.S. Pat. No. 6,855,734 (WO0051596), patent application US 20040033223 (WO02062322) and WO2004049095 (PCT/IB 02/04923), the content and claims of which are incorporated and claimed in the present application, as well as the publications “Betaine supplementation decreases plasma homocysteine concentrations but does not affect body weight, body composition, or resting energy expenditure in human subjects, Ursula Schwab et al, Am. J Clin Nut 2002; 76:961” in which betaine reduces the blood pressure, especially the diastolic pressure, as well as the publication “Betaine, a promising New Agent for Patients With Non-alcoholic Steatohepatitis: Results of a pilot Study Manal F. Abdelmalek et al, The American Journal of Gastroenterology, Vol. 96, No. 9, 2001” in which betaine enables to treat some forms of hepatitis, it was important to have at his disposal a therapeutic formulation for oral path intended to increase the total bioavailability of the betaine so as to reduce the intake amount, while maintaining constant betaine plasmatic levels for long periods.

The present invention discloses these forms and methods, especially the use of floating forms having a density lower than that of gastric juices. It is expected from floating forms that they are maintained durably above the stomach content and consequently increase the bioavailability of the absorbed active principles in the proximal portion of the tractus. These forms represent also the appropriate vehicle of active principles having to act locally in the stomach, namely at least one betaine.

The claimed pharmaceutical forms can also belong to the class of monolithic hydrophilic matrixes. These matrix forms are non-disintegrating and non-digestible. The homogeneous formulation, contained at the start in a hard gelatin capsule, is prepared from one or more hydrophilic polymers. This type of polymer is progressively hydrated in contact with water, swells and builds a surface gelified barrier. The present invention claims the use of any known polymer used in the state of the art for the preparation an advantageously solid controlled release pharmaceutical form and/or a floating form able to be a matrix or not, intended to release at least one betaine after oral administration, preferably a controlled release floating form. At least one betaine as active principle is released from the matrix, in an extended and controlled manner, essentially by diffusion and erosion at the surface. The wording “betaine and/or betaines” in the present specification refers to betaines disclosed and claimed in U.S. Pat. No. 6,855,734 (WO0051596), patent application US 20040033223 (WO02062322) and WO2004049095 (PCT/IB 02/04923) of the inventor, the texts and claims of which are incorporated and claimed the present specification by reference. The betaines as well as combinations of betaines between them, and the combinations of betaines with other molecules disclosed in these applications, can, in the field of the present invention, be formulated, manufactured, synthesized, combined and presented according to the invention in a floating form, with or not controlled release.

The controlled release floating form can also be a bi-layered form, a first layer comprising at least one betaine, while another layer is intended or adapted to increase the floating of said form, said other layer being for example converted in a layer ensuring a floating when in contact with gastric medium or fluid or juices. The controlled release floating form can also be multi-layered, i.e. comprising several layers of at least one betaine separated, for example the one from the other, by several layers intended to increase the floating of said form, said later layers presenting the same or different dissolution indexes and/or reacting at different pH. In a particular aspect of the invention these different layers can contain one or more other therapeutic agent other than betaine, and optionally several other therapeutic agents. These therapeutic agents can be with controlled or immediate release and/or in a form floating or not.

The floating form, comprising at least one release layer of at least one betaine and at least another layer intended to increase the buoyancy of said form, is intended to increase the gastric residence time of at least one betaine so that said betaine is substantially released in the gastro intestinal tractus for an extended period.

According to an aspect of the invention, the buoyant form will comprise any physiologically acceptable polymer, preferably a hydrophilic polymer, which in contact with liquids and/or gastric fluids will form a gelatinous barrier and/or a gelified barrier, the density of which will be lower to that of the gastric fluids and/or the gastric content. The composition intended to release at least one betaine can be in the form of capsules, micro capsules, granules, tablets, liquids, gels, flakes and combinations thereof.

According to an aspect of the invention, the floating form will comprise any physiologically acceptable polymer which can be separated from the betaine by a nonionic barrier film. According to a detail of said embodiment of the invention, the floating form comprises one or more non-ionic barrier, especially one or more barrier films extending between the one or more physiologically acceptable polymers and the betaine or betaine containing support (such as a core).

According to an aspect of the invention, the floating form will comprise several polymers and/or galenic forms in which the active product or products are dispersed in a mixture of two or more types of polymers.

According to an aspect of the invention, the floating form will comprise any excipient known by the skilled man in the art which enables a better gastro intestinal absorption, such as bicarbonate salts, sodium salts, phosphate salts, etc., as well as their precursors, esters and pharmaceutically acceptable salts.

According to another aspect of the invention, the betaines can be synthesized with these excipients, known in the state of the art, for increasing the gastro intestinal absorption as well as the bioavailability.

According to another aspect of the invention, the betaines can be synthesized with these excipients, known in the state of the art, for increasing the adhesion to the gastro intestinal coating.

### MANNERS TO REDUCE INTO PRACTICE THE INVENTION

In the present invention, controlled released floating forms for releasing at least one betaine after oral administration can use in a non limitative way forms and techniques disclosed in the following documents incorporated herein by reference:
The forms disclosed in U.S. Pat. Nos. 4,871,548, 4,767,627; 5,443,843; 5,007,790; 5,582,873; 4,851,232 and U.S. Pat. No. 6,120,803 (WO9907342). In said documents, the hydroxy alkylated celluloses are used as swelling agents in order to increase the residence time. These forms can be used according to the invention so as to release on a controlled manner at least one betaine,
Another possibility for extending the residence time in the stomach is to produce floating forms. These forms float on the stomach contents and, as the pylorus is located in the lowest portion of the stomach, they are not discharged in the small intestine for a very long period. Various processes are known for producing such forms. Thus, it is possible to incorporate substances which have a low density such as for example fats, oils or waxes. Such forms are disclosed in U.S. Pat. No. 4,814,179 (EP198769), U.S. Pat. No. 4,424,235 a continuation of application Ser. No. 301,498 filed Sep. 14, 1981, now abandoned, U.S. Pat. No. 3,834,347 and GB1546448 (BE 839604). These forms can be used in the present invention so as to release on a controlled manner at least one betaine.
The mixtures of U.S. Pat. No. 4,702,918 (DE 3527852) which discloses fatty mixtures which are placed in a capsule and are heated for solidification. These forms can be used in the present invention for release in a controlled manner at least one betaine.
The coated forms such as those produced in U.S. Pat. No. 4,814,179 in which the forms are cooled, shaped and dried. These forms can be used in the present invention for release in a controlled manner at least one betaine.
Another method consists in the use of gases coming from carbonic acid salts. This means that the salts will be incorporated in the dosage forms within a gel or a substance adapted to form a gel after administration. After being exposed to gastric acid, CO₂ is formed which inflates the form, reduces the density of the dosage form and causes its buoyancy. In this case, physiologically tolerated salts such as, for example citric acid, tartaric acid etc. can be incorporated. These preparations are placed in hard or soft gelatin capsules and are disclosed for example in the documents GB 2283172 and U.S. Pat. No. 5,888,540 (GB 2283171). According to the invention, forms disclosed in said documents are suitable for a controlled release of at least one betaine, in particular glycine betaine.
The production of tablets and granules as disclosed in U.S. Pat. No. 6,090,411 (WO9945887) and U.S. Pat. No. 4,167,558 can also be used. The preparation of bilayered tablets as disclosed in U.S. Pat. No. 4,140,755 can further be used. These tablets produce a gel after dissolution, said gel floating in the stomach and releasing the active principles, i.e. according to the present invention at least one betaine. According to the invention, forms disclosed in said documents are suitable for a controlled release of at least one betaine, in particular glycine betaine.
The preparations in which the layers forming the coverings are applied on a core suitable to release CO₂. In certain cases, the covering as such contains a carbonic acid salt. Such preparations are disclosed in U.S. Pat. No. 4,844,905 (EP235718), U.S. Pat. No. 4,101,650 and WO9949868 and can act to release on a controlled manner at least one betaine. According to the invention, forms disclosed in said documents are suitable for a controlled release of at least one betaine, in particular glycine betaine.
Powders which comprise the active agent, a hydrocolloid, a pH dependant polymer, a binder, all of these being placed in a capsule, are disclosed in U.S. Pat. No. 5,169,638. According to the invention, forms disclosed in said documents are suitable for a controlled release of at least one betaine, in particular glycine betaine.
U.S. Pat. No. 5,232,704 discloses forms consisting of two layers, a first containing the active agent while the other causes the floating. Such a system can act for releasing at least one betaine and comprises a layer containing the active agent and another causing the floating. This system can also be applied to multi layered forms in which several layers causing the floating can be used for the betaine by coating successively several layers of at least one betaine or comprising at least one betaine. For example, one layer of betaine coats a floating layer and is coated with one floating layer. In order to have some immediate release, one layer of betaine can only be coated with an enterosoluble layer, and not with a floating layer. The gel forms, the foams and microcapsules containing air such as described in U.S. Pat. No. 6,280,744 (WO9625950), U.S. Pat. No. 5,626,876 (EP326816) and U.S. Pat. No. 6,312,726 (WO9505809) can also act in the present invention. These forms can be used according to the invention for releasing on a controlled manner at least one betaine.
The gel forms, the foams and microcapsules containing air such as described in U.S. Pat. No. 6,280,744 (WO9625950), EP 326816 and U.S. Pat. No. 6,312,726 (WO9505809) can also act in the present invention. According to the invention, forms disclosed in said documents are suitable for a controlled release of at least one betaine, in particular glycine betaine.
U.S. Pat. No. 6,517,866 (WO9901121) discloses effervescent pharmaceutical preparation comprising one or more effervescent excipients and several individual units comprising a pharmaceutically active component and possibly excipients, in which the units are provided with a system causing the floating, said system comprising at least two coating layers, the one generating a gas and the other being a barrier layer comprising the generated gas. Such a system can act for releasing at least one betaine. According to the invention, forms disclosed in said documents are suitable for a controlled release of at least one betaine, in particular glycine betaine.
WO02102415 discloses floating pharmaceutical composition, which comprises one or more therapeutic agents, here at least one betaine, a gelified envelope prepared from Sativum Lepidium seeds, one or more absorption activators, one or more gas generating compounds, and pharmaceutically acceptable excipients. The absorption activators can be xantham gum, karaya gum as well as similar compounds. The composition intended to release at least one betaine can be in the form of capsules, tablets, liquid, gel, flakes, and/or combinations of said forms. This composition enables the absorption of a betaine essentially in the upper part of the gastro intestinal tractus. According to the invention, forms disclosed in said documents are suitable for a controlled release of at least one betaine, in particular glycine betaine,
U.S. Pat. No. 6,635,279 (EP1138320) discloses a mixture of polyvinyl acetate and polyvinylpyrrolidone, as well as excipients which are able to be used so as to form, according to the present invention, the production of a floating form with controlled release of at least one betaine. These forms can be prepared by simple processes and show exceptional mechanical strengths. According to the invention, forms disclosed in said documents are suitable for a controlled release of at least one betaine, in particular glycine betaine.
In the present invention, the floating forms comprising several coated compartments with one or several polymers and generating a pulsatile release such as disclosed in ACCUDEP® cores (Delsys Corp.) can be used for increasing the residence time in the gastro intestinal tractus, of at least one betaine. These forms can be used in the present invention so as to release in a controlled manner at least one betaine.

None of these documents teaches a controlled release form floating or suitable to become floating or buoyant, said form being intended to release on a controlled manner at least one betaine as therapeutic active agent (especially for the treatment of cardiovascular troubles, thrombotic troubles, etc. and/or for preventing such troubles), after oral administration.

None of these documents teaches a buoyant controlled release form intended to increase the total bioavailability of at least one betaine as therapeutic active agent, after oral administration.

None of these documents claims a floating controlled release form intended to increase the total bioavailability of at least one betaine as therapeutic active agent, after oral administration.

None of these documents teaches a floating controlled release form intended to increase the residence time in the gastro intestinal tractus of at least one betaine.

None of these documents claims a floating controlled release form intended to control and/or to increase in function of the time the release of at least one betaine towards the blood flow.

None of these documents claims a floating controlled release form intended to control and/or to increase the released amount of at least one betaine towards the blood flow.

### BRIEF DESCRIPTION OF THE INVENTION

The invention relates to an oral control release formulation for releasing at least one betaine after oral administration to a human, said formulation comprising at least one pharmaceutically acceptable means ensuring an at least partial floating of the formulation releasing at least one betaine, in the gastro-intestinal tractus.

The invention has thus among others as subject matter an oral control release formulation for releasing at least one betaine after oral administration, characterized in that said formulation comprises or is associated to at least pharmaceutically acceptable means ensuring a floating of at least one betaine in the gastro-intestinal tractus.

Advantageously, the pharmaceutically acceptable floating means is a substantially water insoluble means.

Preferably, the pharmaceutically acceptable floating means is a substantially insoluble in the gastric medium.

More specifically, the pharmaceutically acceptable floating means is a means substantially insoluble at least in the pH range comprised between 3 and 7.5, advantageously between 2 and 7.5, preferably between 1 and 7.5.

According to an embodiment, the formulation is for human use, the pharmaceutically acceptable floating means is a means suitable to pass the human transit without or substantially without degradation. Advantageously, the pharmaceutically acceptable floating means comprise fibers adapted for passing the human transit without or substantially without degradation.

According to another embodiment, the floating means comprises a support, advantageously substantially spherical, able to swell in the presence of water.

According to a further embodiment, the pharmaceutically acceptable floating means comprises substantially spherical support swelling in the presence of water.

According to a detail of another embodiment, the formulation comprises solid micro-spheres containing betaine, preferably glycine betaine, said micro-spheres being coated with at least one layer of a hydrophilic polymer or copolymer swelling in contact with human gastric medium, but insoluble in human gastric medium. For example, the formulation comprises solid micro-spheres containing glycine betaine, said micro-spheres being coated with at least one layer of a hydrophilic polymer or copolymer swelling in contact with human gastric medium, but insoluble in human gastric medium.

Advantageously, for each micro-sphere, a controlled release layer that is substantially non swelling in gastric medium is located between the betaine micro-sphere and the hydrophilic polymer or copolymer swelling in contact with human gastric medium.

According to an advantageous embodiment, the formulation comprises solid micro-spheres containing betaine, said micro-spheres being coated with at least one layer of a hydrophilic polymer or copolymer swelling in contact with human gastric medium with a swelling rate of at least 2, preferably of at least 4, most preferably between 5 and 20, but insoluble in human gastric medium.

According to a specific embodiment, the formulation comprises solid micro-spheres containing betaine, said micro-spheres being coated with at least one layer of a hydrophilic polymer or copolymer swelling in contact with human gastric medium, but insoluble in human gastric medium, and in which each micro-sphere is provided with at least one water impermeable barrier layer for protecting at least the hydrophilic swelling polymer layer, said barrier layer being selected among the group consisting of layers soluble in human gastric medium, layers at least partially degradable in human gastric medium and combinations thereof.

According to an advantageous embodiment, the formulation comprises an efficient amount of at least one means ensuring a floating of at least one betaine in the gastro intestinal tractus, so as to ensure the floating of at least 50% by weight of the betaine in the gastro-intestinal tractus for at least 3 hours after the oral administration, preferably for at least 6 hours after the oral administration, most preferably for at least 9 hours after oral administration, especially for at least 12 hours after oral administration.

According to an advantageous embodiment, the formulation comprises an efficient amount of at least one means ensuring a floating of at least one betaine in the gastro intestinal tractus, so as to ensure the floating of at least 85% by weight of the betaine in the gastro-intestinal tractus for at least 3 hours after the administration.

According to another advantageous embodiment, the formulation comprises an efficient amount of at least one means ensuring a floating of at least one betaine in the gastro intestinal tractus, so as to ensure the floating of at least 75% by weight of the betaine the gastro-intestinal tractus for at least 3 hours after the oral administration, preferably for at least 6 hours after the oral administration.

According to an example, the formulation comprises a physiologically acceptable polymer or copolymer adapted, when in contact with the gastric medium, to form a system having a density lower than the density of the gastric medium, advantageously lower than 1, such as lower than 0.9, lower than 0.8.

For example, the physiologically acceptable polymer or copolymer is selected to form a system selected from the group consisting of gelatinous systems, gelified systems and combinations thereof, when in contact with the gastric medium.

Most specifically, the physiologically acceptable polymer or copolymer is a water insoluble hydrophilic polymer or copolymer, said polymer or copolymer being insoluble in human gastric medium.

According to specific embodiments, the formulation has a form selected in the group consisting of capsules, microcapsules, spheroids, liquids, gels, flakes and combinations thereof, said form being advantageously placed in an envelope soluble in contact to the gastric medium.

According to a detail of one specific embodiment, the formulation comprises at least one therapeutically active agent other than betaine.

Said other active agent are for example one or more active agents known for treating patient suffering from or at risk from suffering from at least one trouble selected from cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof.

Advantageously, the weight ratio betaine/therapeutically active agent other than betaine is comprised between 0.1 and 100, preferably between 1 and 100, most preferably between 2 and 50, such as 5, 10, 15, 20, 25, 30, 40 and 50.

According to a specific embodiment, the formulation further comprises at least one therapeutically active agent selected from the group consisting of clopidogrel, the salts thereof, the esters thereof, aspirin, the salts thereof, the esters thereof and combinations thereof.

Advantageously, the formulation comprises an effective amount of less than 200 mg of said therapeutically active agent selected from the group consisting of clopidogrel, the salts thereof, the esters thereof, aspirin, the salts thereof, the esters thereof and combinations thereof. Especially, the formulation comprises clopidogrel bisulfate. The human efficient amount of clopidogrel or aspirin can be reduced. For example, the formulation of the invention are effective with doses of less than 75 mg clopidogrel base (such as with 15 mg up to 50 mg) or with less than 100 mg aspirin (expressed in its acid form), advantageously less than 75 mg, preferably less than 50 mg.

According to another embodiment, the formulation comprises betaine containing particles selected from the group consisting of microparticles, nanoparticles and mixtures thereof. Advantageously, the betaine containing particles are selected from the group consisting of uncoated matrix particles, coated matrix particles and mixtures thereof.

The invention further relates to unit oral dosage form comprising:
a first oral control release formulation for releasing at least one betaine after oral administration to a human, said formulation comprising at least one pharmaceutically acceptable means for ensuring an at least partial floating of the formulation releasing at least one betaine in the gastro-intestinal tractus, said first formulation having advantageously one or more characteristics of the formulation of the invention disclosed here above, and
a second oral formulation for releasing at least one therapeutically active agent different from the betaine released from the first oral controlled formulation.

Said other therapeutically active agent is one or more active agents known for treating patient suffering from or at risk from suffering from at least one trouble selected from cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof. According to an embodiment, the other active agent is clopidogrel (or a pharmaceutically acceptable salt, especially the bisulfate salt, or a pharmaceutically acceptable ester thereof) or aspirin (or a salt or an ester thereof).

The unit dosage form is for example a capsule containing a first formulation in the form of a tablet, granules, micro-spheres, etc., while the other formulation is also in the form of a tablet, granules, micro-spheres, etc. Advantageously a barrier coating is used for separating the two formulations.

According to a specific embodiment, the second formulation is the PLAVIX® formulation, preferably in a smaller dosage form than the 75 mg dosage form.

The second oral formulation can be a controlled release form, such as a delayed forms, extended forms, stepwise release forms (step by step), floating forms, etc. and combinations thereof.

The invention still relates to a unit oral dosage form comprising:
a first oral control release formulation for releasing at least one betaine after oral administration to a human, said formulation comprising at least one pharmaceutically acceptable means for ensuring an at least partial floating of the formulation releasing at least one betaine in the gastro-intestinal tractus, and
a second oral formulation for releasing at least one therapeutically active agent, said second formulation having a different release profile than the controlled release profile of the first oral control release formulation.

For example, two different types of betaine are used, a first type in the first formulation and another type in the second formulation.

According to another example, the same betaine is used in the two formulations, but said formulations having different release profile, for example the second having a more delayed release profile.

The invention relates also to the use of at least one means ensuring a floating of at least one betaine in the gastro-intestinal tractus for the preparation of an oral controlled release formulation of betaine.

Advantageously, the formulation is as disclosed here above for the formulation of the invention.

Advantageously, said formulation comprises at least one pharmaceutically acceptable means ensuring an at least partial, preferably substantially complete floating of the formulation releasing at least one betaine, in the gastro-intestinal tractus, for at least 3 hours after oral administration. Preferably, an efficient amount of at least one means ensuring a floating of at least one betaine in the gastro intestinal tractus is used, so as to ensure the floating of at least 50% by weight of the betaine in the gastro-intestinal tractus for at least 3 hours after the oral administration, such as for at least 6 hours after the oral administration, most preferably for at least 9 hours after oral administration, especially for at least 12 hours after oral administration.

According to a specific embodiment, an efficient amount of at least one means ensuring a floating of at least one betaine in the gastro intestinal tractus is used, so as to ensure the floating of at least 85% by weight of the betaine in the gastro-intestinal tractus for at least 3 hours after the administration.

According to another specific embodiment, an efficient amount of at least one means ensuring a floating of at least one betaine in the gastro intestinal tractus is used, so as to ensure the floating of at least 75% by weight of the betaine in the gastro-intestinal tractus for at least 3 hours after the oral administration.

According to a detail of the use of the invention, the use is adapted for the preparation of a formulation comprising a betaine, advantageously glycine betaine, and having at least one characteristic selected from the group consisting of:
Increasing the residence time in the gastro-intestinal tractus of at least one betaine
Control and/or increase in function of the time of the release of at least one betaine towards the blood flow
Control and/or increase of the released amount of at least one betaine towards the blood flow
Control and/or increase of the bioavailability of at east one betaine, and
Combinations of said characteristics.

The invention further relates to a method for treating a human suffering of at least one trouble selected from the group consisting of cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyperhomocysteinemia, pain and combinations thereof, in which at least one oral formulation is timely administered to said patient, whereby said oral formulation is an oral control release formulation for releasing at least one betaine after oral administration to a human, said formulation comprising at least one pharmaceutically acceptable means ensuring an at least partial foaling of the formulation releasing at least one betaine, in the gastro-intestinal tractus, for at least 3 hours after oral administration.

Advantageously, at least one pharmaceutically acceptable floating means of the oral formulation is ensuring a substantially complete floating of the formulation releasing at least one betaine, in the gastro-intestinal tractus, for at least 3 hours after oral administration. The formulation has advantageously one or more characteristics of the formulation of the invention.

The invention still relates to a method for treating a human at risk from suffering of at least one trouble selected from the group consisting of cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof, in which at least one oral formulation is timely administered to said patient, whereby said oral formulation is an oral control release formulation for releasing at least one betaine after oral administration to a human, said formulation comprising at least one pharmaceutically acceptable means ensuring an at least partial floating of the formulation releasing at least one betaine, in the gastro-intestinal tractus, for at least 3 hours after oral administration.

The formulation has advantageously one or more characteristics of the formulation of the invention.

The invention further relates to polymeric micro- and nanoparticles (such as micro and nanocapsules) containing at least a betaine. Such micro- and nanoparticles shield the encapsulated betaines from the external harsh conditions and/or favor the uptake by intestinal cells. Said micro- and preferably nanoparticles containing at least a betaine can be advantageously be used in the formulation of the invention, as well as in unit dosage forms of the invention.

### BRIEF DESCRIPTION OF THE DRAWING

FIG. 1 shows a betaine liposome (not drawn to scale), comprising a single lipid layer membrane composed of hydrogenated soy phosphatidyl and a second lipid layer, said layers separating the internal aqueous compartment from the external medium. Betaine is encapsulated in the internal medium. Polymer groups (linear segments of polyethylene glycol) are grafted to the liposome surface. The polymer also extends from the inner monolayer of the membrane.

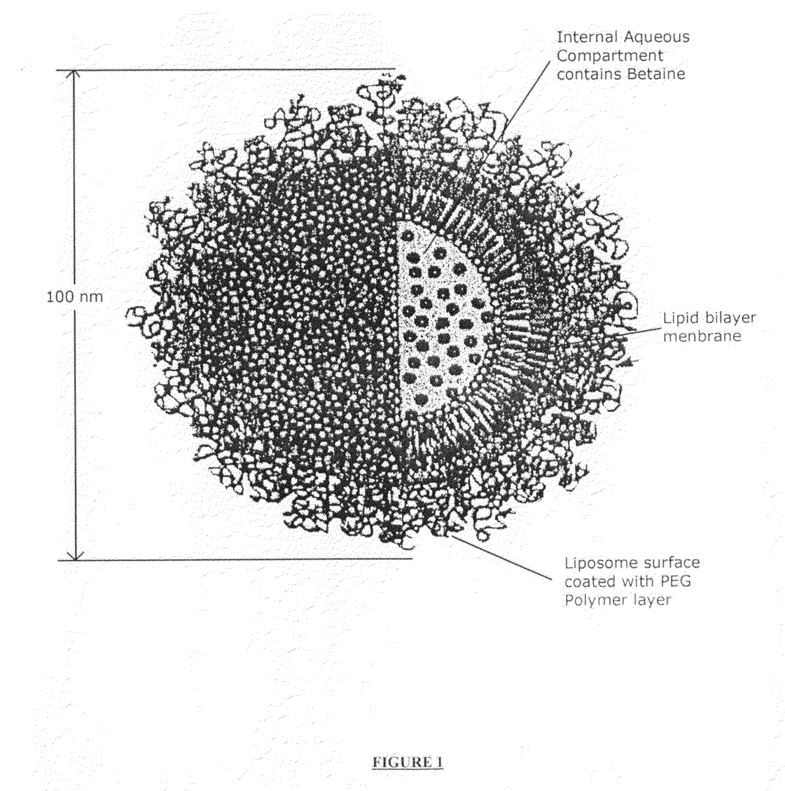

FIG. 2 shows the profile of floatability versus time of a buoyant capsule size N° 1 in a simulated gastric medium.

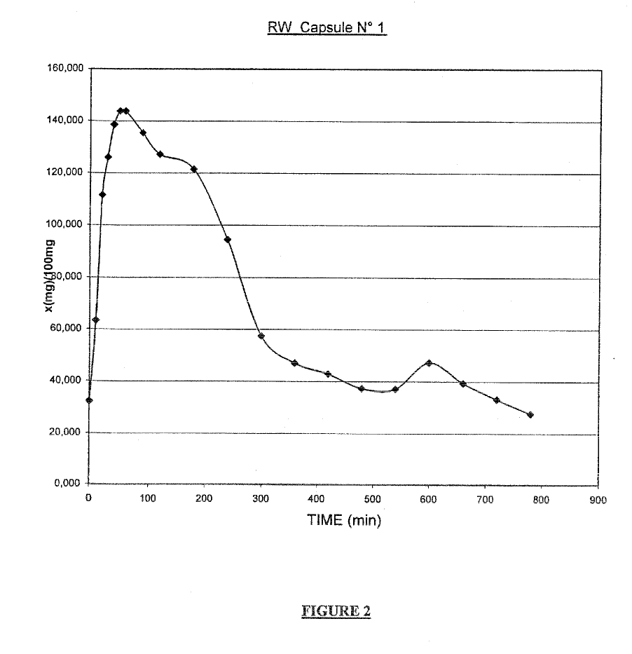

FIG. 3 shows the profile of floatability versus time of a buoyant capsule size N° 5 in a simulated gastric medium.

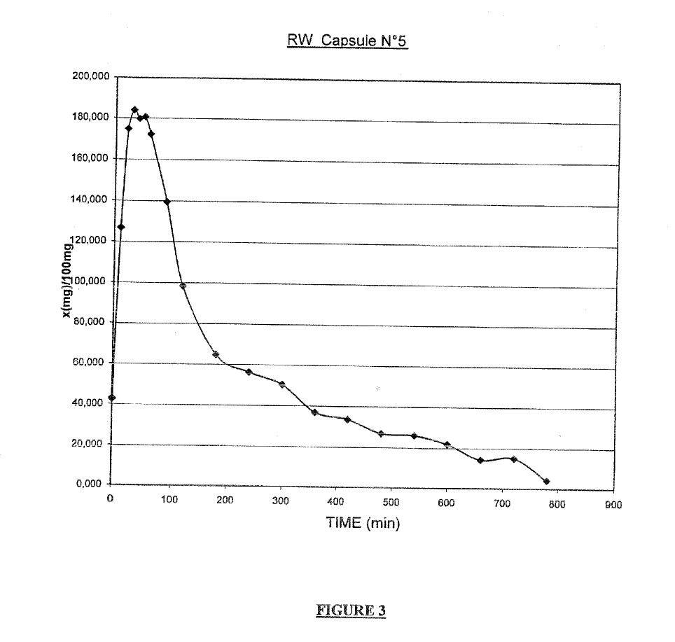

### GENERAL DESCRIPTION OF EMBODIMENTS

The invention has thus among others as subject matter an oral control release formulation for releasing at least one betaine after oral administration, characterized in that said formulation comprises or is associated to at least pharmaceutically acceptable means ensuring a floating of at least one betaine in the gastro-intestinal tractus.

Advantageously, the pharmaceutically acceptable floating means is a substantially water insoluble means, especially insoluble in the gastric medium.

More specifically, the pharmaceutically acceptable floating means is a means substantially insoluble at least in the pH range comprised between 3 and 7.5, advantageously between 2 and 7.5, preferably between 1 and 7.5. When the formulation is for human use, the pharmaceutically acceptable floating means is a means suitable to pass the human transit without or substantially without degradation, for example fibers adapted for passing the human transit without or substantially without degradation.

For example, the floating means comprises a support, advantageously substantially spherical, able to swell in the presence of water.

According to a detail of an embodiment, the formulation has the form of solid micro-spheres containing betaine, in particular glycine betaine, said micro-spheres being coated with at least one layer of a hydrophilic polymer able to swell in gastric medium, but insoluble in gastric medium. The micro-spheres of betaine (before their coating) have for example a size comprised between 50 μm and 5 mm, advantageously between 100 μm and 2 mm, for example 250 μm to 1.5 mm, in particular between 500 μm and 1 mm. The dry weight ratio hydrophilic polymer/betaine micro-sphere is advantageously comprised between 0.5 and 10, advantageously between 1 and 8, for example between 2 and 5.

Advantageously, for each micro-sphere, a layer that is substantially non swelling and controlled release in gastric medium is located between the betaine micro-sphere and the layer able to swell in gastric medium. This enables to control the release of betaine in the polymer layer able to swell once said layer forms a gel around the betaine sphere. According to an advantageous embodiment, the hydrophilic polymer able to swell has a swelling rate of at least 2, advantageously at least 4, preferably comprised between 5 and 20. The swelling rate is defined as the ratio between the maximal volume of 1 gram of polymer in water at a pH of 7 and the volume of polymer in dry condition.

According to an advantageous detail, each micro-sphere is associated to at least one water impermeable barrier layer for protecting at least the hydrophilic swelling polymer layer, said barrier layer being soluble in water, especially for pH lower than 5, or partially degradable in gastric medium. Such a barrier layer enables to avoid a swelling of the hydrophilic polymer before the micro-spheres reach at least the stomach of the patient. When the layer is soluble or degradable, it is advantageously such that it ensures a barrier effect avoiding the swelling of the hydrophilic polymer for at least 30 seconds in water with a pH of 5 and at 20° C.

By degradable layer, it is also meant a layer suitable to swell, such as a layer based on wax or oil, in particular vegetal oil or derivatives of such waxes or oils.

Advantageously, for human, said formulation comprises or is associated to an efficient amount at least one means ensuring a floating of at least one betaine in the gastro intestinal tractus, so as to ensure the floating of at least 50% by weight of the betaine in the gastro-intestinal tractus for at least 3 hours, preferably for at least 6 hours, in particular for at least 9 hours, more specifically for at least 12 hours after the administration. According to a preferred form, said formulation comprises or is associated to an efficient amount of at least one means ensuring the floating of at least 75% by weight of the betaine in the gastro-intestinal tractus for at least 3 hours, preferably for at least 6 hours after the administration. According to an even more specific embodiment, said formulation comprises or is associated to an efficient amount of at least one means ensuring the floating of at least one betaine in the gastro-intestinal tractus, so as to ensure a floating of at least 85% by weight of the betaine in the gastro-intestinal tractus for at least 3 hours after the administration.

According to a detail of an oral formulation according to the invention, this comprises or is associated to physiologically acceptable polymer, preferably water insoluble hydrophilic polymer insoluble in gastric medium, which in contact with gastric liquids and/or fluids is able to form a form, advantageously gelatinous and/or gelified, the density of which is lower than that of the gastric fluids and/or of the gastric content.

The oral formulation of the invention can have the form of capsules, microcapsules, spheroids, liquids, gels, flakes, paillettes and their combinations, possibly placed in an envelope soluble in contact to the gastric juices.

The invention has still for the subject matter the use of at least one means ensuring a floating of at least one betaine in the gastro-intestinal tractus for the preparation of an oral controlled release formulation of betaine, in particular according to the invention.

In particular, the invention relates to the use of said means for the preparation of a formulation comprising a betaine and having at least one characteristic selected from the group consisting of:
increasing the residence time in the gastro-intestinal tractus of at least one betaine
control and/or increase in function of the time of the passage of at least one betaine towards the blood flow
control and/or increase of the released amount of at least one betaine towards the blood flow
control and/or increase of the bioavailability of at least one betaine, and
combinations of said characteristics.

In the field of the present invention, various approaches have been followed and claimed for increasing the retention of a betaine in the gastro-intestinal system, namely: system equilibrated in a hydrodynamic manner, non effervescent systems, expandable systems, bio-adhesive systems, high density systems, systems and apparatuses retarding the gastric draining and the floating systems.

In a particular aspect of the invention, the pH dependent systems and formulations will be used, as well as the hydrodynamic balanced forms.

The polymers and copolymers alone or in combination used in the present invention can be, on a non limiting way, the followings:

Cellulose and its derivatives, polyamide, acrylic polymer, polyvinylpyrrolidone, polyethylene glycol, polystyrene, polyvinylalcohol, vinyl chloride-vinyl acetate copolymer, ethylene-vinyl acetate copolymer, vinyl chloride-propylene-vinyl acetate copolymer, polyvinylformal, polyvinyl acetate, polybutadiene, polyvinylbutyral, polyamides, polyimides, acrylic polymers, polyesters, polyvinylpyrrolidone, starch, polyethyleneglycols, polystyrene, polyvinylalcohol, myristyl alcohol and stearyl alcohol polymers, polyvinyl acetate, polybutadiene, polyvinylbutyral, vinyl chloride-vinyl acetate copolymer, ethylene-vinyl acetate copolymer, vinyl chloride-propylene-vinyl acetate copolymer, hydrocolloid polysaccharides, polycarbonates, polyacrylate, polymethacrylate, acrylate-methacrylate copolymer, cellulose acetate phtalate, ethyl cellulose and methyl hydroxypropyl cellulose, polyethylene oxide, glyceryl monostearate, sodium myristate, alkyl, substituted cellulose, polyacrylic acid and their mixtures.

High-density formulations with a density higher that 1 can also be used, this in order to increase the gastric residence time. This can be achieved by coating the drug with an inert heavy material such as barium sulphate, zinc oxide, titanium dioxide, etc.

According to an aspect of the invention, polymers can be used in order to increase the residence time in the gastro intestinal tractus of at least one betaine. The betaines can be linked, synthesized and/or combined to said polymers so as to increase the residence time in the gastro-intestinal tractus, and formulated in forms floating or not.

In a particular aspect of the invention, mucosa-adhesive polymers can be used so as to increase the residence time in the gastro-intestinal tractus of at least one betaine. The betaines can be linked, synthesized and/or combined to said polymers so as to increase the residence time in the gastro-intestinal tractus, and formulated in forms floating or not.

Lubricants which can be used are, in a non limiting manner, aluminum stearate, calcium, magnesium, tin, magnesium silicates, magnesium stearates, thixcins, silicones and similar substances and their mixtures.

Possible substances for facilitating the flow which can be used are, in non restrictive manner, talc, silica colloids, starch, microcrystalline cellulose and similar substances and their mixtures.

The binders which can be used are, in a non restrictive way, starches, alginates, carboxymethylcellulose or polyvinylpyrrolidone and similar substances and their mixtures. Bulking agents which can be added are, in a non restrictive way, for example, inorganic bulking agents, such as magnesium and/or aluminium and/or silicium oxides, aluminium, silicon, or titanium carbonate or calcium carbonate, sodium bicarbonate, phosphates, sodium chlorides and similar substances and their mixtures.

In a particular aspect of the invention, calcium carbonate, sodium bicarbonate, phosphates, sodium chlorides, their esters and similar substances and their mixtures can be used in order to increase the bioavailability in the gastro-intestinal tractus of at least one betaine.

In the field of the present invention, the release of at least one betaine can be accelerated by adding polymers which are freely soluble in water, and can also be reduced by adding substances which are very lipophilic substances which are water swelling whereby forming a gel in the pores of the inert matrix and thus blocks the diffusion of this betaine outside said matrix. The examples of substances forming such gels can be, in the field of the present invention, alginates, pectins, galactomannanes, carrageenans, dextrans, curdlan, pullulan, gellan, chitins, gelatin, xanthates, hemicellulose, cellulose derivatives such as methylcellulose, hydroxypropylmethylcellulose, hydroxypropylcellulose, hydroethylcellulose, carboxymethylcellulose, starch derivatives such as carboxymethyl starch, degraded starch, maltodextrins, polyacrylic acid, polymethacrylic acid, methacrylic acid acrylic acid copolymers, polyvinylalcohol, high molecular weight polyethyleneglycols, polyoxyethylene/polyoxypropylene copolymers, high molecular weight polyvinylpyrrolidones and their derivatives. The lipophilic substances include, for example, fatty alcohols such as stearyl alcohol, fatty acids such as stearic acid, glycerides, fatty acid esters and fatty alcohol esters, lipophilic polymers such as ethylcellulose, cellulose acetate, methicrylic ester acrylic ester copolymers, methacrylic ester acrylic acid copolymers, phtalate acetate cellulose, succinate acetate cellulose, phthalate acetate hydroxypropylmethylcellulose, acetate succinate hydroxypropylmethylcelluloe. The water soluble polymers include, for example, polyethylene glycols, polyvinylpyrrolidone or vinylpyrrolidone actate vinyl copolymers.

Coloring agents which can be added are iron oxides, titanium dioxide, triphenylmethane dyes, azo dyes, quinoleine dyes, indigo dyes, carotenoids, opacifying agents such as titanium dioxide or talc for increasing the transparency or for preserving the coloring agents. Substances which increase the permeability of the intestine membrane, known in the art, can also be used in the field of the present invention so as to optimize the transferred amount of at least one betaine in the blood. Absorption activators can be gums, resins, salts, as well as any substance known by the man skilled in the art for this purpose.

According to the invention, means such as described in the present application can be combined, in a non restrictive way, for optimizing the invention and the efficiency of claimed compositions and/or forms.

The forms as claimed in the present invention can increase the residence time in the gastro intestinal tractus of at last one betaine, for 3 hours, 6 hours, 12 hours, 24 hours, 48 hours, 72 hours, 96 hours, one week, one month.

In one embodiment, the forms as claimed in the present invention can improve betaine plasmatic profiles when providing higher, sustained and more constant plasmatic levels of betaine in a human.

According the invention, the claimed forms augment patients' compliance, especially for long term treatments.

In one embodiment, the forms as claimed in the present invention are intended for once a day dosing.

Gums such as these coining from gellan, carragheen gum, tragacanth gum, ghatti gum, glucomannan, guar gum, acacia gum, locust bean gum, xanthan gum, veegum, gellan gum andtragacanth derivatives, cellulose like compounds, hydroxy propyl methyl cellulose, hydroxypropyl cellulose, carboxymethyl cellulose and its derivatives, pectins, lignin, chitins and its derivatives, acrylic acids, aguar, gelatin, polyvinyl alcohol, carbomers such as carbopols, or their combinations can be used as bio-adhesives and/or substances improving the passage of at least one betaine from the gastro-intestinal tractus towards the blood circulation.

The substances generating a gas can generate carbon dioxide or sulphur dioxide in contact with gastric liquid. Examples of gas generating substances which can be used in the invention include carbon dioxide generating substances, such as calcium carbonate or sodium glycine carbonate, bicarbonate such as the sodium hydrogen carbonate or potassium hydrogen carbonate, sulphur dioxide generating compounds such as sodium sulphite, sodium bisulphite, sodium metabisulphite and similar substances.

The gas generating compound reacts by simple contact with the gastric fluid for generating carbon dioxide or sulphur dioxide which is confined in the matrix or the gel of the floating form, which under this effect increases of volume and floats above the gastric fluids and releases on a controlled manner at least one betaine.

According to the invention, the floating composition can contain soluble or insoluble diluting agents, preferably in water. Examples of water soluble diluting agents include in a non-restrictive way, lactose, sucrose, mannitol and similar substances. Examples of insoluble diluting agents which can be used in the present invention include, but are not restricted to, dibasic calcium phosphate, starch and microcrystalline cellulose.

Pharmaceutically acceptable binders such as polyvinylpyrrolidone 30/90, hydroxypropyl methylcellulose and hydroxypropylcellulose can be used in the present formulation. Further to the ingredients mentioned here above, colloidal silicon dioxide (AEROSIL® 200) can be used as lubricant, in the matrix. Preferably, magnesium stearate, stearic acid, talc and colloidal silicon dioxide, alone or in combination can be used. Antioxidants can also be included.

Tablets can be coated with a film which is rapidly dissolved so as to form a water soluble polymer such as hydroxypropyl methylcellulose, acrylate, ethyl cellulose and/or water soluble excipients.

According to a further aspect of the invention, the forms can be multi compartments with different disintegration profiles and/or times. These compartments with different disintegrating profiles and/or times can, in case of saturation of the absorption site at the level of the gastro intestinal tractus for at least one betaine, to control the amount and/or the moment when this betaine will be released so that the optimal absorption is achieved on said site. In an aspect of the invention, these compartments can replace the successive layers solvents of betaine and agents ensuring the floating and/or the adhesion in the gastro intestinal tractus.

The terms “betaine” and/or “betaines” used in the present invention refer to compounds selected from the group consisting of lipidic betaines, betaine lipids, and/or betaines of formula (CH₃)₃N⁺—(CH₂)ₙ—COO⁻ with n an integer from 1 to 5, (preferably glycine betaine n=1), their pharmaceutically acceptable salts, their esters, their precursors and their mixtures.

The terms “lipidic betaines” and “betaine lipids” refer to betaine lipids and/or lipidic betaines which are structural elements of the membranes commonly found in ferns, mosses, mycetes, mushrooms, amoebas, eukaryotes, wild plants and algae. The betaine lipids are ether bound non-phosphoric glycerolipids, which look by their general structure to the most commonly known phosphatidylcholine. The most common glycerolipids contain a diacyl glycerol portion to which a polar group is attached. This polar group can be carbohydrate portion such as in the glycero phospholipids, the most common lipid class of the animals. The betaine lipids represent a third class of glycero lipids in which a quaternary alcohol amine is bound in an ether bond to the diacylglycerol portion. They can be obtained by extraction, biosynthesis or synthesis. The betaine lipids diacylglyceryl-O-4′-(N,N,N-trimethyl)-β-homoserine and a similar isoform, the diacylglyceryl-O-2′-(hydroxymethyl) (N,N,N-trimethyl)-β-alanine are the most common.

According to a particular aspect of the invention, these lipidic forms can be preferred due to their better bioavailability.

According to a particular aspect of the invention, the betaines can be combined or synthesized with lipids, oils or fats or their mixtures.

According to a particular aspect of the invention, the floating form can be a liquid form, said liquid form swelling in contact with juices and/or acids of the gastro intestinal environment. This swelling modifies the density of said liquid form so as to ensure its floating.

According to an aspect of the invention, the pharmaceutical forms which can be hydro-dynamically balanced or not and which can be carried out belong to the class of monolithic hydrophilic matrixes. These matrix forms are not disintegrating and non digestible. The homogenous formulation, contained in a hard gelatin capsule, is prepared from one or more hydrophilic polymers.

The described forms do not limit the field of the invention and can in particular be combined for carrying out the invention.

The invention further relates to polymeric micro- and nanoparticles as such (such as microcapsules, nano capsules) containing at least a betaine. Such micro- and nanoparticles shield the encapsulated betaines from the external harsh conditions and/or favor the uptake by intestinal cells. Said micro- and preferably nanoparticles containing at least a betaine can be used for the preparation of various dosage forms, such as tablets, granules, etc. or for filling gelatin or other capsules. Said dosage forms can be non-floating or floating. In an embodiment, said micro- and/or nano-particles or capsules are used in the formulation of the invention, as well as in unit dosage forms of the invention.

The micro- and nanoparticles are provided with a protection coating. Said coating is advantageously an enteric coating.

Polymeric particles will isolate the encapsulated betaines from the external medium therefore protecting them from dissolution and dilution in the gastric medium allowing, then, their uptake by enterocytes. After absorption, polymeric particles can less or more rapidly degrade according to a kinetic profile depending on the nature of the polymer, thus providing a sustained and controlled release of the drug. The size of the micro and the nano particles as well as the nature of the polymer will be designed, according to the invention, to be from several nanometers to several micrometers as to augment their uptake by enterocytes, for releasing one or more betaines in the blood stream and/or for augmenting their bioavailability.

Polymeric particles used for drug delivery are defined as colloidal systems made of solid polymers that may be classified according to their size and preparation processes. The term microparticle designates entities or systems larger than 1 μm, but advantageously of less than 100 μm, preferably less than 50 μm, whereas nanoparticles are submicronic particles smaller than 1 micrometer.

Micro- and nanocapsules are composed of a polymeric wall containing a core, solid, semi-solid or not, advantageously a liquid inner core or a core suitable to become liquid or semi-liquid after administration (for example betaine is mixed with a pharmaceutically acceptable compound having a melting point between 30 and 60° C., advantageously about 35 to 40° C.), where the betaines are entrapped, while micro- and nanospheres are advantageously made of a solid polymeric matrix in which the drug can be dispersed, said micro- and nano spheres being advantageously coated. The betaines may be either adsorbed at the surface of the polymer or encapsulated within the particle. Particles may be produced by polymerization of synthetic monomers, or dispersion of synthetic polymers or natural macromolecules. According to an embodiment, the micro- and/or nanoparticles, especially the micro- and/or nanocapsules, are dispersed in a pharmaceutically acceptable medium, such as a matrix or a pharmaceutically acceptable gum or a pharmaceutically acceptable liquid. When dispersed in a liquid or a gel, the micro or nanoparticles will be advantageously provided with a barrier layer, for example a water insoluble layer, so as to avoid a dissolution or release of betaine before the oral administration.

When dispersed in a liquid or semi-liquid composition, the formulation can have the form of a micro- or nano dispersion or emulsion. In such a case a dispersing agent and/or a tensioactive agent are advantageously used. Such a dispersing or tensioactive agent can be amphoteric, ionic, non ionic, cationic. Examples of tensioactive are: sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, POLOXAMER®, etc. Mixtures of surfactants are also suitable.

The micro- and/or nanoparticles, especially the micro- and nanocapsules, containing betaine can also be tableted, so as to form a tablet with a slower release rate than the release rate of the micro- or nanoparticles, the micro- or nanoparticles being disintegrated slowly from the tablet.

The micro- or nanoparticles, especially the micro- or nanocapsules, containing a betaine, can also be placed in a capsule, such as a hard gelatin capsule, a soft gelatin capsule, a hydropropyl cellulose capsule, etc.

The micro- or nanoparticles or capsules, especially their outer protective coating (such as an enteric coating) are advantageously submitted to a curing step, for example at a temperature comprised between 50° C. and 110° C., such as between 60° C. and 90° C.

According to the invention, different types of polymeric particles can be used such as but non limited: nanospheres, nanospheres prepared by the simple or double emulsion technique, nanocapsule where the betaines are entrapped, nanospheres where the betaines are adsorbed, extrusion-spheronization, etc.

Possible nature of the polymers and preparation methods for polymeric particles comprising betaines are given in the following table:

| | | Nature of |
|---|---|---|
| Polymer | Method | particles |
| PLA | Emulsion-evaporation | Spheres |
| | Emulsion-diffusion | Spheres |
| | Salting out | Spheres |
| | Desolvation | Capsules |
| | Interfacial precipitation | Capsules |
| PLGA | Emulsion-evaporation | Spheres |
| | Interfacial precipitation | Capsules |
| PACA | Interfacial polymerization | Capsules |
| | Emulsion polymerization | Spheres |
| Chitosan | Emulsion-diffusion | Spheres |
| EUDRAGIT ® | Emulsion-diffusion | Spheres |
| | Interfacial precipitation | Capsules |
| P(MMA-g-EG) | Suspension | Spheres |
| | polymerization | |
| HPMC-AS | Emulsion-evaporation | Spheres |
| Poly FA:SA | Phase inversion | Spheres |
| Insulin | Desolvation | Spheres |
| | Cross-linking | |
| LHRH-n- | Copolymerization | Spheres |
| butylcyanoacrylate | | |
| Cyclosporin A | Emulsion-precipitation | Spheres |
| Derivatized amino | Self-aggregation | Spheres |
| acids | | |
| Soybean protein | Self-aggregation | Spheres |
| hydrolysates | | |

In said table, the meanings of the abbreviations are:
PLA: poly(lactic acid)
PLGA: poly(lactic-co-glycolide)
PACA: poly(alkylcyanoacrylate)
EUDRAGIT®: poly(methylacrylic acid-co-methylmethacrylate)
P(MAA-g-EG): poly(methacrylic-g-ethylene glycol)
FA:SA: fumaric acid:sebacic acid
HPMC-AS: hydroxypropylmethylcellulose acetate succinate
LHRH: luteinizing hormone releasing hormone

According to the invention the polymers and the methods of the table can be substituted them between, and/or can be combined.

Particle formation by polymerization reactions (emulsion-polymerization) can use polymers such as poly-methylmethacrylate, poly-alkyl cyanoacrylate, and poly-methylidenemalonate. The water insoluble monomer is dispersed in an aqueous phase and the polymerization is induced and controlled by addition of a chemical initiator or by variations in physical parameters such as pH or gamma-radiation in the presence or the absence of surfactants to stabilize the emulsion. Betaines are entrapped in the polymeric wall when added to the polymerization medium or adsorbed on preformed particles afterwards.

In one embodiment the betaines can be derivatized to form a hydrophobic complex. The derivatization process can use any of the compounds known by the skilled man to be efficient and physiologically acceptable.

In another embodiment, a double emulsion technique is used in which an aqueous solution of one or more betaines is first emulsified in an organic solution of the polymer. This primary emulsion is then poured into a large volume of water with or without surfactant. In a specific embodiment, the double emulsion technique can entrap one or more betaine in alveolar structures.

In still another embodiment, the particles are prepared from a preformed polymer according to the spray-drying method where one or more betaine are solubilized and/or dispersed in an organic solution of the polymer to be nebulized in a hot air flow. The solvent is almost instantly evaporated and dried microparticles are readily recovered. This method can be suitable for augmenting particles passage through enterocytes and/or for augmenting their adherence to enterocytes allowing a slow diffusion of one or more betaines.

In an other embodiment one or more betaine is polymerized or grafted on a polymer or copolymer.

In an other embodiment one or more betaines can be submitted to the technique of co-polymerization with for example n-butylcyanoacrylate radicals followed by the precipitation of the co-polymer to form nanoparticles.

In an other embodiment one or more betaine can be used in oily suspensions and/or emulsion for improving the entrapment in nano- and microcapsules.

Micro- and nanocapsules may be prepared by interfacial polymerization from alkylcyanoacrylate monomers. One or more betaine and the monomer are dissolved and/or dispersed in a mixture of ethanol and oil under magnetic stirring into an aqueous phase. Capsules may also be obtained from preformed polymer, based on a dessolvation process.

In an other embodiment the methods based on the emulsification-diffusion process can be used for the preparation method for capsules with one or more betaine core.

In one embodiment, when the particles are formed, spheres or capsules, a purification step is needed to remove the additives necessary for the manufacturing or to separate unincorporated betaines. It is used as well as a concentration technique. This may be achieved by centrifugation or ultracentrifugation depending on the size of the particles, by filtration and/or centrifugal filtration and/or cross flow filtration and/or gel permeation and/or dialysis and their combinations.

Betaine oligonucleotide nanoparticles can represent effective colloidal drug carriers.

Nanoparticles were obtained, using betaine and phosphorothioate in water. However, the colloidal solution in water could be stabilized by polyethylenglycol 20000 (PEG), which also led to an increase of stability in cell medium.

In one embodiment, one or more Betaine as a cationic compound can be used as a potential penetration enhancer in the gastro-intestinal tract for phosphodiester antisense oligonucleotides.

In one embodiment, one or more Betaine as a cationic compound can be used as a potential penetration enhancer in the gastro-intestinal tract for glycosaminoglycans.

In one embodiment, one or more Betaine as a cationic compound can be used as a potential penetration enhancer in the gastro-intestinal tract for insulins.

In one embodiment, one or more betaine is encapsulated in liposomes, micro-emulsions, or biodegradable particles, and the modification of one or more betaine through the attachment of chemical moieties.

In one embodiment, one or more betaine is encapsulated in liposomes comprising advantageously a single lipid layer membrane and optionally a coating polymer.

In one embodiment, one or more betaine is encapsulated in liposomes comprising advantageously multiple lipid layer membrane (such as a hi layer membrane) and optionally a coating polymer.

In one embodiment, one or more betaine is modified through the attachment of chemical moieties for augmenting its passage through intestinal cells.

In one embodiment, one or more betaine is modified through the attachment of chemical moieties for augmenting its passage through intestinal cells, such chemical moieties selected from the pharmaceutical acceptable salts of a betaine and/or esters of betaine and/or combinations thereof.

In one embodiment, one or more betaine is synthesized with chemical moieties for augmenting their passage through intestinal cells.

In one embodiment, the attachment and/or synthesis of chemical moieties to one or more betaine form a complex comprising the drug (one or more betaine) and the prodrug (one or more chemical moieties). This drug/prodrug complex can partially or completely dissociate on intestinal cells layer and/or in intestinal cells layer and/or after its passage in the blood stream.

In one embodiment, Betaine lipo-spheres can be used. Lipospheres are an aqueous micro-dispersion of water insoluble, spherical microparticles (0.2 to 100 μm in diameter), each consisting of a solid core of hydrophobic triglycerides and betaine particles that are embedded with phospholipids on the surface. Such lipospheres may use vehicles made of microparticulates or colloidal carriers composed of lipids, carbohydrates or synthetic polymer matrices. Liposomes, the most widely studied of these vesicles, can be formulated to include a variety of compositions and structures that are potentially non-toxic, degradable and non-immunogenic. Formulations of one or more betaine can be realized with polylactic acid, lecithin, iophendylate and phosphatidylcholine and cholesterol, respectively.

### USES OF THE INVENTION

Due to the better plasmatic profiles of betaine they provide, the formulations of the present invention can be particularly suitable for treating and/or preventing and/or alleviating one or more troubles provoked by one or more diseases and/or troubles selected from the group consisting in:

Cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof.

### EXAMPLES

### Example 1

### Floating Betaine Tablet

1.6 kg betaine, 1.6 kg of a mixture comprising polyvinyl acetate and polyvinylpyrrolidone in the proportion 8:2 (KOLLIDON® SR) and 0.02 kg of magnesium stearate have been passed through a sieve of 0.8 mm, mixed in a TURBULA® mixer for 10 minutes, then compacted in an eccentric press KORSCH® EKO so to manufacture biplanar tablets with a weight of 650 mg. The compression strength was 3.04 kN.

The tablets were floating immediately after adding a simulated gastric fluid. The floating continued for 38 hours.

Release profile of a floating tablet

| Time (h) | Amount released (%) |
|---|---|
| 0 | 0 |
| 1 | 15.6 |
| 2 | 23.1 |
| 4 | 37.7 |
| 8 | 52.3 |
| 12 | 65.0 |
| 18 | 83.6 |
| 24 | 98.2 |

### Example 2

### Hydroxypropylmethylcellulose Floating Tablet of Betaine

1.6 kg betaine, 1.6 kg of hydroxypropylmethylcellulose (METHOCEL® K100) and 0.02 kg of magnesium stearate have been passed through a 0.8 mm sieve, mixed in a Turbula mixer for 10 minutes and then compacted in an eccentric press KORSCH® EKO so to manufacture biplanar tablets with a weight of 650 mg. The compression strength was 2.05 kN.

The tablets were floating immediately after adding a simulated gastric fluid. The floating continued for 24 hours.

Release profile of a floating tablet

| Time (h) | Amount released (%) |
|---|---|
| 0 | 0 |
| 1 | 12.7 |
| 2 | 21.4 |
| 4 | 36.2 |
| 8 | 58.3 |
| 12 | 66.9 |
| 18 | 79.5 |
| 24 | 92.6 |

### Example 3

### Acrylic Ester Copolymer Floating Tablet of Betaine

1.6 kg betaine, 1.6 kg EUDRAGIT®, RS and 0.02 kg magnesium stearate have been passed through a 0.8 mm sieve, mixed in a TURBULA® mixer for 10 minutes and then compacted in an eccentric press KORSCH® EKO so to manufacture biplanar tablets with a weight of 650 mg. The compression strength was 5.05 kN.

The tablets were floating immediately after adding a simulated gastric fluid. The floating continued for 37 hours.

Release profile of a floating tablet

| Time (h) | Amount released (%) |
|---|---|
| 0 | 0 |
| 1 | 13.9 |
| 2 | 22.6 |
| 4 | 40.3 |
| 8 | 61.8 |
| 12 | 75.9 |
| 18 | 84.9 |
| 24 | 98.0 |

### Example 4

### Preparation of a Controlled Release Granule

A mixture comprising 38% of compactable EUDRAGIT® RPL RTM, 60% anhydrous betaine, 1% talc stearate and 1% magnesium is prepared. After treatment in a TURBULA®T2C mixer, the mixture was compacted by means of an EKO apparatus of KORSCH® at 40,000 N/cm² and then converted into granules by means of a granulator ERWEKA® TG II S. The granules have then been dressed by means of a vibrator keeping only the fractions having a diameter between 100 and 150 μm. The so obtained granules can be used in various preparations, in particular capsules, tablets or gels.

### Example 5

Betaine powder (particle size of less than 250 μm, in particular of less than 100 μm) has been mixed with oil or wax or an alcohol solid at room temperature, for example coconut oil or stearylic alcohol. For preparing this mixture, the oil, alcohol or wax was melted so as obtain a liquid form. Betaine in powder was added to the molten oil, wax or alcohol. The weight ratio betaine/oil or wax or alcohol was varied between 0.05 and 2 (ratios lower than 1 being preferred such as 0.2, 0.3, 0.4 and 0.5).

The liquid mixture is converted in micro-spheres, for example by spraying or by extrusion-spheronization, etc. The so prepared micro-spheres have a size of 500 μm, 750μm and 1 mm.

These micro-spheres have been coated in one or more steps, with a layer of a hydrophilic polymer swellable in water, but insoluble in gastric medium (for example acrylic polymer or copolymer such as EUDRAGIT®). The dry weight ratio hydrophilic polymer/betaine micro-sphere was varied between 1 and 10.

The so obtained balls have then been provided with a thin layer of a polymer insoluble in water, but soluble or degradable in gastric medium, for example a EUDRAGIT® polyacrylate coating.

### Example 6

### Buoyant Capsules of Betaine

### Products Used

Capsules size n° 1: CAPSUGEL® lot n°: 31012061
Capsules size n° 5: CAPSUGEL® lot n°: 30197
Xanthan gum: CRETE® lot n°: 04E11FN
Sodium hydrogenocarbonate: MERCK® lot n°: K27484023

### Excipients Composition

### Diluting Mix:

| | |
|---|---|
| Xantham gum | 75 g |
| Sodium hydrogenocarbonate | 75 g |

### Capsules Content

| | |
|---|---|
| Anhydrous betaine | 75% |
| Diluting mix | 25% |

### Operating Mode:

The work was performed under an inflated bubble of dry air (±15 to 20% of relative humidity).

Capsules size n° 1 Anhydrous Betaine 260 mg

Capsules are filed by compressing 75% anhydrous betaine (260 mg) supplemented with 25% diluting mix (85 mg Xanthan gum/hydrogenocarbonate Na).

Capsules size n° 5: Anhydrous Betaine 70 mg (for small animals experimental uses)

Capsules are filed with 75% anhydrous betaine supplemented with 25% diluting mix (Xanthan gum/hydrogenocarbonate Na).

### Weight Uniformity

Method:

The containing of each capsule is determined by subtracting the average weight of 20 empty capsules from the total weight of each capsule.

Results:

Capsules size n°1

| Total weight | Contained masse | Variation |
|---|---|---|
| in g | in g | in % |
| 0.43295 | 0.35875 | 2.24 |
| 0.42288 | 0.34868 | 0.62 |
| 0.41408 | 0.33988 | 3.13 |
| 0.41306 | 0.33886 | 3.42 |
| 0.44340 | 0.36920 | 5.22 |
| 0.42875 | 0.35455 | 1.05 |
| 0.41340 | 0.33920 | 3.33 |
| 0.42320 | 0.34900 | 0.53 |
| 0.46773 | 0.39353 | 12.15 |
| 0.40264 | 0.32844 | 6.40 |
| 0.43800 | 0.36380 | 3.68 |
| 0.43169 | 0.35749 | 1.89 |
| 0.46039 | 0.38619 | 10.00 |
| 0.42464 | 0.35044 | 0.12 |
| 0.41698 | 0.34278 | 2.31 |
| 0.41916 | 0.34496 | 1.68 |
| 0.39946 | 0.32526 | 7.29 |
| 0.41328 | 0.33908 | 3.36 |
| 0.41585 | 0.34165 | 2.63 |
| 0.41982 | 0.34562 | 1.50 |

Average weight = 0.35087 g
CV = 4.90%

The contain weight=capsule total weight minus average weight of 20 empty capsules (0.074198 g).

Capsules size n° 5

| Total weight | Contained masse | Variation |
|---|---|---|
| in g | in g | in % |
| 0.11465 | 0.08832 | 3.00 |
| 0.11360 | 0.08727 | 4.15 |
| 0.12490 | 0.09857 | 8.26 |
| 0.11363 | 0.08730 | 4.12 |
| 0.11942 | 0.09309 | 2.24 |
| 0.11500 | 0.08867 | 2.61 |
| 0.11801 | 0.09168 | 0.69 |
| 0.11485 | 0.08852 | 2.78 |
| 0.11567 | 0.08934 | 1.88 |
| 0.11812 | 0.09179 | 0.81 |
| 0.11482 | 0.08849 | 2.81 |
| 0.11716 | 0.09083 | 0.24 |
| 0.11936 | 0.09303 | 2.17 |
| 0.11950 | 0.09317 | 2.33 |
| 0.11412 | 0.08779 | 3.58 |
| 0.12075 | 0.09442 | 3.70 |
| 0.11991 | 0.09358 | 2.78 |
| 0.12234 | 0.09601 | 5.45 |
| 0.11766 | 0.09133 | 0.31 |
| 0.11425 | 0.08792 | 3.44 |

Average weight = 0.09105 g
CV = 3.48%

The contain weight=capsule total weight minus average weight of 20 empty capsules (0.026333 g).

Disintegration:

In buffer 1.2-0.05M—ionic strength 0.2, with the disks −37° C.

| Capsules size n° 1 | Capsules size n° 5 |
|---|---|
| 5′23 | 3′45 |
| 6′39 | 4′06 |
| 7′39 | 4′18 |
| 8′03 | 4′35 |
| 8′31 | 4′49 |
| 9′11 | 6′54 |
| Mean: 7′34 | Mean: 4′44 |

Accordingly, a capsule (such as CAPSUGEL®) filled only with betaine powder will release immediately, its betaine content in the medium. After the start of the disintegration of the capsule, the betaine in contact with the medium will dissolve.

### Floatability of Betaine Buoyant Capsules.

Medium: HCl 0.1 N+0.05% polysorbate 20

### Results:

| | Capsules size n° 5 | Capsules size n° 1 |
|---|---|---|
| Time (min.) | Beads RW (%) | Beads RW (%) |
| 0 | 42,857 | 32,258 |
| 10 | 126,891 | 63,364 |
| 20 | 174,790 | 111,521 |
| 30 | 184,034 | 126,037 |
| 40 | 179,832 | 138,710 |
| 50 | 180,672 | 143,779 |
| 60 | 172,269 | 143,779 |
| 90 | 139,496 | 135,484 |
| 120 | 98,319 | 127,189 |
| 180 | 64,706 | 121,429 |
| 240 | 56,303 | 94,470 |
| 300 | 50,420 | 57,373 |
| 360 | 36,975 | 47,005 |
| 420 | 33,613 | 42,857 |
| 480 | 26,891 | 37,327 |
| 540 | 26,050 | 37,097 |
| 600 | 21,849 | 47,235 |
| 660 | 14,286 | 39,401 |
| 720 | 15,126 | 33,180 |
| 780 | 4,202 | 27,650 |

The maximum buoyancy arises appreciatively 1 hour after the beginning of the test either for Capsule size N° 1 or Capsule size NC 5. RW stands for Resulting Weight.

What I claim is:

1. An oral control release formulation for releasing at least one betaine after oral administration to a human, said formulation having a density greater than 1 and comprising at least one pharmaceutically acceptable means for ensuring buoyancy of the formulation releasing at least one betaine in the gastro-intestinal tractus.

2. The formulation of claim 1, in which the pharmaceutically acceptable buoyancy means is a water insoluble means.

3. The formulation of claim 1, in which the pharmaceutically acceptable buoyancy means is a gastric insoluble means.

4. The formulation of claim 1, in which the pharmaceutically acceptable buoyancy is a means insoluble at least in the pH range comprised between 3 and 7.5.

5. The formulation of claim 1, in which the pharmaceutically acceptable buoyancy is a means insoluble at least in the pH range comprised between 2 and 7.5.

6. The formulation of claim 1, in which the pharmaceutically acceptable buoyancy is a means insoluble at least in the pH range comprised between 1 and 7.5.

7. The formulation of claim 1, in which the pharmaceutically acceptable buoyancy means is a means suitable to pass the human transit without degradation.

8. The formulation of claim 7, in which the pharmaceutically acceptable buoyancy means comprises fibers adapted for passing the human transit without degradation.

9. The formulation of claim 1, in which the pharmaceutically acceptable buoyancy means comprises a support swelling in the presence of water.

10. The formulation of claim 1, in which the pharmaceutically acceptable buoyancy means comprises spherical support swelling in the presence of water.

11. The formulation of claim 1, which comprises solid micro-spheres containing betaine, said micro-spheres being coated with at least one layer of a hydrophilic polymer or copolymer swelling in contact with human gastric medium, but insoluble in human gastric medium.

12. The formulation of claim 11, in which for each micro-sphere, a controlled release layer that is non swelling in gastric medium is located between the betaine micro-sphere and the hydrophilic polymer or copolymer swelling in contact with human gastric medium.

13. The formulation of claim 1, which comprises solid micro-spheres containing glycine betaine, said micro-spheres being coated with at least one layer of a hydrophilic polymer or copolymer swelling in contact with human gastric medium, but insoluble in human gastric medium.

14. The formulation of claim 1, which comprises solid micro-spheres containing betaine, said micro-spheres being coated with at least one layer of a hydrophilic polymer or copolymer swelling in contact with human gastric medium with a swelling rate of at least 2, but insoluble in human gastric medium.

15. The formulation of claim 1, which comprises solid micro-spheres containing betaine, said micro-spheres being coated with at least one layer of a hydrophilic polymer or copolymer swelling in contact with human gastric medium with a swelling rate of at least 4, but insoluble in human gastric medium.

16. The formulation of claim 1, which comprises solid micro-spheres containing betaine, said micro-spheres being coated with at least one layer of a hydrophilic polymer or copolymer swelling in contact with human gastric medium with a swelling rate comprised between 5 and 20.

17. The formulation of claim 1, which comprises solid micro-spheres containing betaine, said micro-spheres being coated with at least one layer of a hydrophilic polymer or copolymer swelling in contact with human gastric medium, but insoluble in human gastric medium, and in which each micro-sphere is provided with at least one water impermeable barrier layer for protecting at least the hydrophilic swelling polymer layer, said barrier layer being selected among the group consisting of layers soluble in human gastric medium, layers degradable in human gastric medium and combinations thereof.

18. The formulation of claim 1, which comprises an efficient amount of at least one means ensuring buoyancy of at least one betaine in the gastro intestinal tractus, so as to ensure the floating of at least 50% by weight of the betaine in the gastro-intestinal tractus for at least 3 hours after the oral administration.

19. The formulation of claim 1, which comprises an efficient amount of at least one means ensuring buoyancy of at least one betaine in the gastro intestinal tractus, so as to ensure the floating of at least 50% by weight of the betaine in the gastro-intestinal tractus for at least 6 hours after the oral administration.

20. The formulation of claim 1, which comprises an efficient amount of at least one means ensuring buoyancy of at least one betaine in the gastro intestinal tractus, so as to ensure the floating of at least 50% by weight of the betaine in the gastro-intestinal tractus for at least 9 hours after oral administration.

21. The formulation of claim 1, which comprises an efficient amount of at least one means ensuring buoyancy of at least one betaine in the gastro intestinal tractus, so as to ensure the floating of at least 50% by weight of the betaine in the gastro-intestinal tractus for at least 12 hours after oral administration.

22. The formulation of claim 1, which comprises an efficient amount of at least one means ensuring buoyancy of at least one betaine in the gastro intestinal tractus, so as to ensure the floating of at least 85% by weight of the betaine in the gastro-intestinal tractus for at least 3 hours after the administration.

23. The formulation of claim 1, which comprises an efficient amount of at least one means ensuring buoyancy of at least one betaine in the gastro intestinal tractus, so as to ensure the floating of at least 75% by weight of the betaine in the gastro-intestinal tractus for at least 3 hours after the oral administration.

24. The formulation of claim 1, which comprises an efficient amount of at least one means ensuring buoyancy of at least one betaine in the gastro intestinal tractus, so as to ensure the floating of at least 75% by weight of the betaine in the gastro-intestinal tractus for at least 6 hours after the oral administration.

25. The formulation of claim 1, which comprises a physiologically acceptable polymer or copolymer forming, when in contact with the gastric medium, a system having a density lower than the density of the gastric medium.

26. The formulation of claim 25, in which the physiologically acceptable polymer or copolymer is selected to form a system selected from the group consisting of gelatinous systems, gelified systems and combinations thereof, when in contact with the gastric medium.

27. The formulation of claim 25, in which the physiologically acceptable polymer or copolymer is a water insoluble hydrophilic polymer or copolymer, said polymer or copolymer being insoluble in human gastric medium.

28. The formulation of claim 1, which comprises a physiologically acceptable polymer or copolymer forming, when in contact with the gastric medium, a system having a density lower than 1.

29. The formulation of claim 1, which has a form selected in the group consisting of capsules, microcapsules, spheroids, liquids, gels, flakes and combinations thereof.

30. The formulation of claim 1, which has a form selected in the group consisting of capsules, microcapsules, spheroids, liquids, gels, flakes, paillettes and combinations thereof, said form being placed in an envelope soluble in contact to the gastric medium.

31. The formulation of claim 1, which comprises at least one therapeutically active agent other than betaine.

32. The formulation of claim 31, in which the weight ratio betaine/therapeutically active agent other than betaine is comprised between 0.1 and 100.

33. The formulation of claim 1, which further comprises at least one therapeutically active agent selected from the group consisting of clopidogrel, the salts thereof, the esters thereof, aspirin, the salts thereof, the esters thereof and combinations thereof.

34. The formulation of claim 33, which comprises an effective amount of less than 200 mg of said therapeutically active agent selected from the group consisting of clopidogrel, the salts thereof, the esters thereof, aspirin, the salts thereof, the esters thereof and combinations thereof.

35. The formulation of claim 1, which comprises betaine containing particles selected from the group consisting of microparticles, nanoparticles and mixtures thereof.

36. The formulation of claim 35, in which the betaine containing particles are selected from the group consisting of uncoated matrix particles, coated matrix particles and mixtures thereof.

37. An unit oral dosage form comprising:
a first oral control release formulation for releasing at least one betaine after oral administration to a human, said formulation comprising at least one pharmaceutically acceptable means for ensuring an at least partial floating of the formulation releasing at least one betaine in the gastro-intestinal tractus, and
a second oral formulation for releasing at least one therapeutically active agent different from the betaine released from the first oral controlled formulation.

38. An unit oral dosage form comprising:
a first oral control release formulation for releasing at least one betaine after oral administration to a human, said formulation comprising at least one pharmaceutically acceptable means for ensuring an at least partial floating of the formulation releasing at least one betaine in the gastro-intestinal tractus, and
a second oral formulation for releasing at least one therapeutically active agent, said second formulation having a different release profile than the controlled release profile of the first oral control release formulation.

39. Use of at least one means ensuring buoyancy of at least one betaine in the gastro-intestinal tractus for the preparation of an oral controlled release formulation of betaine having a density of greater than 1.

40. The use of claim 39, for the preparation of an oral control release formulation for releasing at least one betaine after oral administration to a human, said formulation comprising at least one pharmaceutically acceptable means ensuring buoyancy of the formulation releasing at least one betaine, in the gastro-intestinal tractus, for at least 3 hours after oral administration.

41. The use of claim 39, in which an efficient amount of at least one means ensuring buoyancy of at least one betaine in the gastro intestinal tractus is used, so as to ensure the floating of at least 50% by weight of the betaine in the gastro-intestinal tractus for at least 3 hours after the oral administration.

42. The use of claim 39, in which an efficient amount of at least one means ensuring buoyancy of at least one betaine in the gastro intestinal tractus is used, so as to ensure the floating of at least 50% by weight of the betaine in the gastro-intestinal tractus for at least 6 hours after the oral administration.

43. The use of claim 39, in which an efficient amount of at least one means ensuring buoyancy of at least one betaine in the gastro intestinal tractus is used, so as to ensure the floating of at least 50% by weight of the betaine in the gastro-intestinal tractus for at least 9 hours after oral administration.

44. The use of claim 39, in which an efficient amount of at least one means ensuring buoyancy of at least one betaine in the gastro intestinal tractus is used, so as to ensure the floating of at least 50% by weight of the betaine in the gastro-intestinal tractus for at least 12 hours after oral administration.

45. The use of claim 39, in which an efficient amount of at least one means ensuring buoyancy of at least one betaine in the gastro intestinal tractus is used, so as to ensure the floating of at least 85% by weight of the betaine in the gastro-intestinal tractus for at least 3 hours after the administration.

46. The use of claim 39, in which an efficient amount of at least one means ensuring buoyancy of at least one betaine in the gastro intestinal tractus is used, so as to ensure the floating of at least 75% by weight of the betaine in the gastro-intestinal tractus for at least 3 hours after the oral administration.

47. The use of claim 39 for the preparation of a formulation comprising a betaine and having at least one characteristic selected from the group consisting of:
Increasing the residence time in the gastro-intestinal tractus of at least one betaine
Control and/or increase in function of the time of the release of at east one betaine towards the blood flow
Control and/or increase of the released amount of at least one betaine towards the blood flow
Control and/or increase of the bioavailability of at east one betaine, and
Combinations of said characteristics.

48. A method for treating a human suffering of at least one trouble selected from the group consisting of cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof, in which at least one oral formulation is timely administered to said patient, whereby said oral administration is an oral control release formulation for releasing at least one betaine after oral administration to a human, said formulation comprising at least one pharmaceutically acceptable means ensuring buoyancy of the formulation releasing at least one betaine, in the gastro-intestinal tractus, for at least 3 hours after oral administration.

49. A method for treating a human at risk from suffering of at least one trouble selected from the group consisting of cardiovascular diseases, atherosclerosis, hypertension, diabetes, cardiac diseases, atrial fibrillation, blood coagulation troubles, Raynaud's disease, Alzheimer disease, vascular dementia, Parkinson disease, memory troubles, intermittent claudication, blood circulation troubles, legs circulation troubles, peripheral arterial disease, peripheral arterial occlusive disease, haemodialysis troubles, renal diseases, liver diseases, metabolic syndrome, syndrome X, ocular troubles, pulmonary hypertension, angina pectoris, stroke, scleroderma, deep venous thrombosis, air travel troubles, polyps, nasal polyps, portal hypertension, sepsis, bleeding troubles, cancer, cancer therapy, chemotherapy, Idiopathic Thrombocytopenic Purpura, restinosis, stent restinosis, thrombocytopenia, psoriasis, inflammation, endothelial dysfunction, sexual dysfunctions, hemorrhoids, fatigue, pneumonia, asthma, trauma, surgery, inflammation, sub-fertility, lactation problems, gut disorders, arthritis and other joint problems, ageing, impaired immune function, burns, malaria, cystic fibrosis, tuberculosis, migraine, neurological problems, schizophrenia, depression, respiratory infections, HIV, muscle soreness, drug intoxication, homocysteine related troubles, homocystinuria, homocysteinemia, hyper-homocysteinemia, pain and combinations thereof, in which at least one oral formulation is timely administered to said patient, whereby said oral formulation is an oral control release formulation for releasing at least one betaine after oral administration to a human, said formulation comprising at least one pharmaceutically acceptable means ensuring buoyancy of the formulation releasing at least one betaine, in the gastro-intestinal tractus, for at least 3 hours after oral administration.
